(19) United States
(12) Patent Application Publication
(10) Pub. No.: US 2023/0072755 A1
(43) Pub. Date: Mar. 9, 2023

(54) MEDICAL DEVICE COMPRISING AN ACELLULAR BIOLOGICAL MATRIX AND AT LEAST ONE POLYMER

(71) Applicant: PH TECH, LE TOURNE (FR)

(72) Inventors: Anthony PERES, DOMPIERRE SUR MER (FR); Guillaume HOFMANSKI, MIEUXCE (FR)

(21) Appl. No.: 17/919,373

(22) PCT Filed: Apr. 16, 2021
(86) PCT No.: PCT/EP2021/059944

§ 371 Date: Oct. 17, 2022

(30) Foreign Application Priority Data
Apr. 17, 2020 (FR) FR2003871

(51) Int. Cl. A61L 27/36; A61L 27/34

(52) U.S. Cl. CPC A61L 27/362; A61L 27/34; A61L 2420/02; A61L 2430/40

(57) ABSTRACT

The invention relates to a medical device comprising at least one acellular biological matrix and at least one polymer, as well as to a method for manufacturing same and to the use thereof as a medical device or as an implant.

### TECHNICAL FIELD

The invention relates to a special medical device comprising a biological matrix, as well as to a method for manufacturing same and to the use thereof.

### PRIOR ART

Many surgical applications require the reinforcement of soft tissues with elements combining mechanical resistance, flexibility, and biological compatibility.

To this end, biological matrices are used more and more for the manufacture of medical devices on account of their biological compatibility. However, in the case of septic or potentially septic surgeries, biological implants can be digested and degraded due to an enzymatic load (collagenases in particular), which makes their use unsuitable for many applications.

A need therefore exists for biological implants having mechanical integrity and optimal reinforcement during tissue reconstruction even in the event of infections.

It is the object of the invention to meet this need.

### SUMMARY OF THE INVENTION

To this end, the invention relates to a medical device comprising at least one acellular biological matrix and at least one polymer, in particular at least one acellular biological matrix that is covered in whole or in part by at least one layer comprising at least one polymer.

Advantageously, the presence of at least one polymer with the acellular biological matrix makes it possible to render the biological matrix resistant to infections while retaining the qualities of the biological matrix in terms of mechanical resistance, flexibility, and biological compatibility. It can thus be used for medical applications, particularly in surgery.

The invention also relates to a method for manufacturing such a medical device. The method comprises the implementation of the following steps:
preparing a cellular biological matrix so as to allow the adhesion of a solution comprising at least one polymer, and
partial or total coating of the acellular biological matrix with a solution comprising at least one polymer.

Other features and advantages will emerge from the detailed description of the invention that follows.

### DETAILED DESCRIPTION OF THE INVENTION

### Definitions

Within the meaning of the invention, the term “acellular” biological matrix is intended to refer to a biological matrix in which the cellular elements have been eliminated through a decellularization process with the aim of destroying and/or removing the cells and their components from the extracellular matrix of the biological matrix while maintaining its structure and properties. In fact, in order for a biological matrix to be implanted in a recipient, it must be decellularized so as to decrease its immunogenicity.

Within the meaning of the invention, the term “allograft” is understood to refer to a biological matrix-a graft-originating from a donor belonging to the same biological species as the recipient.

Within the meaning of the invention, the term “implant” is understood to refer to a medical device used in surgery.

Within the meaning of the invention, the term “biological matrix” is understood to refer to a biomaterial derived from the human or animal species.

Within the meaning of the invention, the term “P4HB” is understood to refer to poly-4-hydroxybutyrate, a specific PHA. It is a homopolymer of a 4-hydroxybutyrate unit.

The term “peel test” is used here to refer to a test making it possible to determine the strength of adhesion between two materials. Each material is placed in pneumatic jaws at a given pressure and then separated at constant speed as specified in the examples.

Within the meaning of the invention, the term “PHA” is understood to refer to polyhydroxyalkanoates, which are biodegradable polyesters.

Within the meaning of the invention, the term “solution” is understood to refer to a homogeneous mixture resulting from the dissolution of one or more solutes in a solvent.

Within the meaning of the invention, the term “suture retention force” or “suture retention strength” is understood to refer to a test for determining the force (N) required to pull a suture out of a specimen.

Within the meaning of the invention, the term “uniaxial tensile strength” is understood to refer to a test for determining the tension prior to the rupturing of the specimen being tested. The properties measured are ultimate tensile strength, breaking strength, and elongation at rupture.

Within the meaning of the invention, the term “viscosity” is understood to refer to a property of resistance to the flow of a fluid for non-turbulent flow.

Within the meaning of the invention, the term “xenograft” is understood to refer to a biological matrix—a graft—originating from a donor belonging to a biological species different from that of the recipient.

### Medical Device

According to a first aspect, the invention therefore relates to a medical device comprising:
at least one acellular biological matrix, and
at least one polymer.

Acellular biological matrices make up a large class of biomaterials that are extracted from grafts of various origins.

Preferably, the biological matrix in the medical device according to the invention is of human and/or animal origin.

According to a particularly suitable embodiment, it is a biological matrix selected from among biological matrices of porcine, bovine, equine, caprine, or fish origin, and mixtures thereof.

The biological matrix according to the invention can be selected from among all animal and/or human biological matrices, preferably from one of the following biological matrices: dermis, intestinal submucosa, aorta, bladder, amniotic membrane, peritoneum, pericardium, dura mater, tendons, bones, cartilage, and mixtures thereof.

The biological matrix of the medical device according to the invention is acellular. There are many known methods for obtaining an acellular biological matrix. The methods used can be enzymatic and/or based on chemical solutions and/or on mechanical processes. The method used must be a method that makes it possible to obtain an acellular biological matrix that is capable of being used in surgery, particularly for the reconstruction of soft tissues.

The acellular biological matrix according to the invention is preferably an acellular biological matrix that has at least one of the following characteristics, even more preferably all of them:
uniaxial tensile strength greater than or equal to 5 N/mm2 for a 5 mm × 50 mm specimen with a thickness taken into account in the calculation. Each end is secured in pneumatic grips lengthwise 1 cm from each edge. The edge spreading rate is 30 mm/min. Uniaxial tensile strength is found by dividing the maximum force (N)/(5 (mm) × thickness (mm)).
suture retention force greater than or equal to 5N for a 1 cm × 1 cm sample using an appropriate suture (4-0 polypropylene-type) threaded 3 mm from the edge of the sample in its center, the opposite side being clamped in a pneumatic grip at approximately 5 mm. The two ends of the suture are clamped in the lower grip. The grip separation speed is 20 mm/min. The maximum force (N) is noted.

Preferably, the biological matrix used has the specifications defined in the applicable standards (“USP official monographs” latest version, ASTM) according to the type of biological matrix used.

The acellular biological matrix of the device according to the invention can have different shapes. It is preferably:
dry, preferably having a residual moisture level of between 10% and 18%, and/or
it has a surface appearance that improves the adhesion of at least one polymer, particularly the adhesion of at least one polymer solution. This surface appearance can be obtained by treatment/preparation of the acellular biological matrix before coating, in particular by chemical and/or mechanical and/or electrochemical and/or physical surface treatment.

The polymer present in the medical device according to the invention can be any type of polymer that is suitable for use as a medical device and particularly as a surgical implant.

The polymer or polymers present in the device according to the invention are preferably selected from among the following polymers: poly(glycolides), poly(lactide-co-glycolides); polylactic acid, polyglycolic acid, poly(lactic acid-co-glycolic acids), polycaprolactones, poly(orthoesters), polyanhydrides, poly(phosphazenes), polyhydroxyalkanoates (including, in particular, P4HB and poly-3-hydroxybutyrate-co-3-hydroxy valerate (PHBV)), polyesters, poly(lactide-co-caprolactones), polycarbonates, tyrosine polycarbonates, polyamides, polyesteramides, poly(dioxanones), poly(alkylene alkylates), polyethers, polyvinyl pyrrolidones or PVP, polyurethanes, polyether esters, polyacetals, polycyanoacrylates, poly(oxyethylene)/poly(oxypropylene) copolymers, polyacetals, polyketals, polyphosphates, polyphosphoesters, polyalkylene oxalates, polyalkylene succinates, polymaleic acids, chitin, chitosan, and mixtures thereof.

According to a particularly suitable embodiment, the device according to the invention comprises at least one PHA, and even more preferably a PHA that is selected from among at least P4HB, the copolymers of P4HB, and mixtures thereof. PHAs are a family of materials produced by many microorganisms. One noteworthy example is U.S. Pat. 6,316,262 by Metabolix, Inc. from Cambridge, MA, USA, which describes a method for obtaining a biological system that enables the production of polyhydroxyalkanoate polymers containing 4-hydroxy acids. Patents US 6,245,537, US 6,623,748, US 7,244,442, and US 8,231,889 also describe methods for preparing PHAs that are suitable for medical use and for medical devices according to the invention.

Preferably, PHA and, in particular, P4HB and/or copolymers thereof have a low level of endotoxins; in particular, they make it possible for levels of below 20 EU per medical device to be achieved.

Preferably, the device comprises at least one acellular biological matrix that is covered in whole or in part by at least one layer comprising at least one polymer.

The device according to the invention can comprise one or more acellular biological matrices, one or more layers of polymer(s), and optionally other constituents. For example, the medical device according to the invention can comprise at least one acellular biological matrix that is covered in whole or in part by at least two layers comprising at least one polymer.

According to a particular embodiment, the medical device consists exclusively of an acellular biological matrix that is covered in whole or in part by a layer comprising at least one polymer.

According to another particular embodiment, the medical device consists exclusively of an acellular biological matrix that is covered in whole or in part by two layers comprising at least one polymer.

The presence of the polymer(s) in the medical device enables optimal reinforcement to be achieved during tissue reconstruction supported by the biological matrix, even in the event of infection.

The polymer layer(s) may comprise one or more channels that make it possible, during use in surgery, to incorporate elements promoting optimal reconstruction of the tissues in which the device is used as an implant (PRP, stem cells, antibiotics, etc.). These channels can be circular or non-circular. They preferably have an internal surface of between 0.007 mm2 and 0.8 mm2. They can be obtained by imprinting a mold in the biological matrix through pressing before coating of the biological matrix. Once the mold has been removed, this is followed by coating and drying.

### Manufacturing Method

The medical device according to the invention can be obtained by any suitable method. In particular, the invention relates to a method for manufacturing a medical device comprising implementation of the following steps:
preparing a cellular biological matrix so as to allow the adhesion of a solution comprising at least one polymer
partial or total coating of the acellular biological matrix with a solution comprising at least one polymer.

Preferably, the step of preparing the cellular biological matrix consists of a chemical and/or mechanical and/or electrochemical and/or physical surface treatment. For example, it can be a treatment by abrasion and/or milling and/or microtexturing and/or laser and/or UV.

The biological matrix must be prepared so as to allow the adhesion of a polymer solution. The properly prepared biological matrix can have different shapes (round, square, circular, irregular, etc.) and be planar or in relief, with channels, with one of the surfaces textured, and have a regular or irregular thickness.

In a preferred embodiment, the biological matrix must be dry or dried so as to have a residual moisture level on the order of from 10% to 18%. The residual moisture level is preferably measured using a Mettler Toledo brand halogen moisture analyzer-type desiccator.

One drying technique used is preferably that of “Loss on Drying” described in USP 41 (“scaffold bovine dermis”):
1 an empty aluminum dish is positioned in the device and taring is carried out,
2 the dish is filled with a sample of 1.0 g +/- 0.2 g cut into 4 mm2 pieces,
3 the heating program at 130° C. is launched
4 when the weight no longer varies over a given time, the result is displayed in %.

Another conventional oven drying method can also be used. In this context, an aluminum dish previously weighed empty is filled with a sample of 5.0 g +/- 0.2 g cut into 4 mm2 pieces. The entire thing is heated at 100° C. for 16 h. It is then weighed and the loss on drying is calculated:
dry matter % = [(weight of the dry extract + dish (g) - weight of dish (g))/g of sample] × 100
moisture % = 100 - dry matter %.

The prepared biological matrix is then used as a support for the application of a polymer solution coating.

Preferably, the solution comprising at least one polymer was obtained previously through solubilization of the dry polymer(s) in at least one solvent, preferably at least one polar solvent.

In fact, the polymer(s) must be preferably converted into a solution that can enable coating using the appropriate solvent.

When the polymer is a PHA and, in particular, P4HB and/or one of its copolymers, the solvent is preferably selected from among the following polar solvents: dichloromethane, chloroform, tetrahydrofuran, dioxane, acetone, and mixtures thereof.

In a preferred embodiment, the P4HB is dissolved in an acetone solution, preferably in a ratio of from 5% to 25% (w/w). The respective amounts are combined, heated, and maintained at a temperature below the boiling point of acetone (approximately 56° C.) until the P4HB dissolves completely and the desired viscosity is obtained, at least 10%, more preferably 15%, and even more preferably 20% (w/w).

Preferably, after solubilization of the polymer(s) in a solvent, the polymer solution is degassed and/or debubbled in order to purge the mixture of air bubbles. Preferably, the solution is placed under vacuum (minimum -1 bar) for the time required for complete degassing/boiling.

The step of coating the acellular biological matrix with a polymer solution is preferably carried out at a temperature less than or equal to the denaturation temperature of the collagen. In a particularly suitable manner, the coating is performed at a temperature between 10 and 60° C., preferably between 10 and 50° C., even more preferably between 20 and 50° C.

The coating can be performed by any suitable means, preferably by solvent casting (coating by casting), spray coating (coating by spraying), dip coating (coating by immersion).

Advantageously, the method according to the invention allows for the direct application of a polymer solution at the desired concentration to a previously prepared acellular biological matrix, and it is not necessary to make a polymer film or sheet beforehand and then place it on the support in order to allow adhesion of the elements to each other by heating.

When it is carried out by “solvent casting,” the coating can be carried out using different technologies: knife, double side, commabar, case knife, engraved roller, 2 rollers, 3 roller combi, microroller, 5 rollers, reverse roller, rotary screen, dipping, slot die, curtain coating, or hot-melt slot die. The simplest method is based on the use of an Elcometer-type casting knife. The biological matrix is placed on a table, and the quantity of polymer solution required as a function of the desired thickness and the surface to be treated is placed on the acellular biological matrix. The gardener knife is then moved over the acellular biological matrix in order to make the coating on the implant uniform. The gardener knife has previously been adjusted to a certain height.

A “coating machine” can be used to automate the operation. The polymer(s) solution is pumped through a slot die in order to be applied to the moving biological implant. In a preferred embodiment, the width of the slot die is 600 mm, and the conveyance speed of the implant is from 1 - 10 m/min. The pumping speed, the conveyance speed, the width of the slot die, and the concentration of the solution can be adjusted in order to obtain an implant with a coating of the desired thickness and width.

When the coating is carried out by “spray coating” or atomization, the polymer solution is pumped up to a nozzle that projects droplets onto the surface to be treated. This technique is particularly advantageous for the treatment of biological matrices having 3D shapes (example: biological implant having a hemispherical, ovoid, tubular shape, or in the form of anatomical breast implants, etc.).

When the coating is performed by means of “dip coating,” or immersion, the part to be treated is dipped in a dissolved, melted, softened, or fluidized powder material in order to cover it with a layer of that material. This technique is particularly advantageous for the treatment of raised and planar biological implants.

After coating, the medical device obtained is composed of a layer of biological matrix that is coated in whole or in part with at least one polymer in solution, and the entire thing is then dried and then preferably pressed in order to obtain a uniform thickness.

In a preferred embodiment, the acellular biological matrices that are coated with the polymer solution are placed in a furnace or oven or heating chamber in order to allow complete evaporation of the solvent. At a temperature between 0° C. and 50° C., between 15 and 40° C., preferably 30° C. plus or minus 5°, so as to prevent excessively rapid evaporation and deformation of the biological matrix.

According to one variant, the acellular biological matrices that have been coated with the polymer solution, optionally before or after drying, are pressed in a hydraulic press for a duration of 30 to 60 s, at between 6 bar and 200 bar, in particular at between 50 and 200 bar and at a temperature between 50° C. and 190° C., in particular between 50° C. and 100° C. Planar biological matrices (and consequently the planar medical devices according to the invention) are treated in a hydraulic press between two plates. Biological matrices (and consequently the medical devices according to the invention) in relief (biological implant-type treated by said method in the form of breast implants in anatomical or hemispherical shapes) can be pressed in molds having the desired imprints (molding machine-type for fabric cup (bra cup press).

### Uses

The medical device according to the invention can be used for any medical application, in particular as an implant, particularly in surgery. It can be used as such or transformed for use in surgeries.

In particular and without limitation, the medical devices according to the invention can be used for the following applications: repair, regeneration, and replacement of soft and hard tissues, healing device, bandage, patch, dressing, dressing for burns, dressing for ulcers, skin substitute, hemostatic, tracheal reconstruction device, organ rescue device, dural substitute, dural patch, nerve guide, nerve regeneration or repair device, hernia repair device, hernia mesh, hernia plug, temporary wound or tissue support device, tissue engineering scaffolding, guided tissue repair/regeneration device, mesh fixation devices, non-stick membrane, adhesion barrier, tissue separation membrane, retention membrane, sling, pelvic floor reconstruction device, urethral suspension device, urinary incontinence treatment device, bladder repair device, bulking or filling device, rotator cuff repair device, meniscus repair device, meniscus regeneration device, guided tissue regeneration membrane for periodontal tissue, anastomosis device, cell-seeded device, cell encapsulation device, controlled release device, drug delivery device, plastic surgery device, breast lift device, mastopexy device, breast reconstruction device, breast augmentation device, breast reduction device, breast reconstruction devices after mastectomy with or without breast implants, rhinoplasty device.

The invention is now illustrated by examples and test results.

### EXAMPLES AND TEST RESULTS

### Example 1: Treatment of the Acellular Biological Matrix

A planar acellular dermal matrix is placed on a digitally controlled machine tool. A carbide bur measuring 5 mm in diameter is mounted on the machine tool. A rotation speed of from 20,000 to 40,000 rpm is used with an advancement of 2 m/min. The depth is variable depending on the desired final thickness. The surfacing can be total or concern only a portion of the implant so as to define shapes.

### Example 2: Treatment of the Acellular Biological Matrix

A planar acellular dermal matrix is placed on a brushing/carding machine. The surface of the implant is thus treated with cards of desired diameter (105 mm, for example) composed of metallic wires (having a diameter of 0.2 mm, for example).

### Example 3: Treatment of the Acellular Biological Matrix

A flat dermal matrix is placed on a Mercier-Turner sander. The surface of the implant is sanded with a 240-grit abrasive.

A section of the abraded implant (6 cm × 6 cm) is coated with approximately 0.02 g/cm2 of P4HB and is pressed for 60 s at 50 bar of pressure and 50° C. (previously heated press plates).

A T-peel test is carried out. Specimens of 2 cm × 6 cm are cut out. The P4HB coating is separated from the biological implant on a section measuring 1.5 cm × 2 cm. The two pieces separated in this manner are placed in the pneumatic grips (45 psi). The section of the coated biological implant is separated at a speed of 25 mm/min. The T-peel force is measured over a standard width of 20 mm and on an average of 5 peaks (loads). The adhesion was stronger on the three specimens that were tested, the coated layer did not manage to be peeled in the test.

### Example 4: Treatment of the Acellular Biological Matrix

An acellular dermal matrix is treated with a high-frequency electrical field of the Telea Biotech-type (4-64 MHz) so as to create cavities and/or perforations 0.6 mm in diameter.

### Example 5: Treatment of the Acellular Biological Matrix

An acellular dermal matrix is treated as specified in example 1. A 2 mm carbide cutter is mounted on the machine tool in order to draw in the matrix channels having a sinusoidal shape along the length of the matrix. Speed and advancement are similar to example 1. The channels are repeated at regular intervals across the width in order to cover part or all of the implant. The depth of the channels depends on the desired final diameter. In another embodiment, an impression can be made so as to press the channels into the matrix.

### Example 6: Example of a Solvent Casting Coating Method

An acellular dermal matrix treated according to example 1 having the dimensions 2 cm × 6 cm, or 12 cm2, is placed on a table. Since the desired amount of P4HB is 0.0164 g/cm2, an 18% P4HB/acetone solution (w/w) is prepared. The required amount is removed and deposited on the implant. The casting knife is then moved translationally on the implant in order to make the thickness of the coating uniform. A coating machine can be used to automate the operation. An acetone/P4HB solution is prepared in order to supply the machine. The solution is pumped through a slot die in order to be applied to the moving planar biological implant. In a preferred embodiment, the width of the slot die is 600 mm, the conveyance speed of the implant is from 1 - 10 m/min.

### Example 7: Example of the Dip Coating Process

An acellular biological matrix treated according to example 4 measuring 4 cm × 6 cm is immersed by a machine (the implant is placed on a vertical crosspiece) into a tank containing an acetone/P4HB solution at the desired concentration. The biological matrix is then extracted from the tank by an upward vertical movement at a speed of 25 mm/min. The density of P4HB obtained by coating is then from 0.02 to 0.04 g/cm2. The parameters (size of the part to be treated, speed of the crosspiece, concentration of the solution) that are required to obtain the desired coating can be ultimately conceived of by those skilled in the art.

### Example 8: Example of Drying and Pressing and Characteristics of the Medical Devices Obtained

A dermal matrix treated according to example 1 is coated with approximately 0.03 g/cm2 of P4HB and is pressed for 30 s at 100 bar of pressure and at 50° C. (previously heated press plates). On three test pieces tested, with an average thickness of 1.35 mm, the maximum average uniaxial tensile strength (UTS) is 26.50 MPa.

The test is carried out on an Instron measurement bench model 3342/L2345. The specimen is cut with a bone shape V cutter-type as described in standard ASTM D-638-5. The piece cut in this manner is introduced by each end into the pneumatic grips of the bench (60 psi), leaving a central portion of 2.5 cm. A speed of 25 mm/min is applied until the part breaks. The uniaxial tensile strength is noted (max. force/sectional area).

A T-peel test is carried out. Specimens of 2 cm × 6 cm are cut out The P4HB coating is separated from the biological implant on a section measuring 1.5 cm × 2 cm. The two pieces separated in this manner are placed in the pneumatic grips (45 psi). The section of the coated biological implant is separated at a speed of 25 mm/min. The T-peel force is measured over a standard width of 20 mm and on an average of 5 peaks (loads). The adhesion was stronger on the three specimens that were tested, the coated layer did not manage to be peeled in the test.

### Example 9: Example of Drying and Pressing and Characteristics of the Medical Devices Obtained

Employing the same conditions as in the preceding tests, a dermal matrix that has been treated according to example 2 is coated with approximately 0.03 g/cm2 of P4HB and then pressed for 60 s at 200 bar and 100° C., resulting in an average UTS max value over three specimens of 27.65 MPa. The average T-peel test cannot be calculated, since the coat is incomplete.

### Example 10

Employing the same conditions as in the preceding tests, a dermal matrix that has been treated according to example 3 is coated with approximately 0.03 g/cm2 of P4HB and then pressed for 30 s at 100 bar and 100° C., resulting in an average UTS max value over three specimens of 33.07 MPa.

1. A medical device comprising at least one acellular biological matrix and at least one polymer.

2. The medical device as set forth in claim 1, characterized in that the acellular biological matrix is covered in whole or in part by at least one layer comprising at least one polymer.

3. The medical device as set forth in claim 2, characterized in that it consists of an acellular biological matrix that is covered in whole or in part by a layer comprising at least one polymer.

4. The medical device as set forth in claim 1, characterized in that the acellular biological matrix is covered in whole or in part by at least two layers comprising at least one polymer.

5. The medical device as set forth in claim 1, characterized in that it consists of an acellular biological matrix that is covered in whole or in part by two layers comprising at least one polymer.

6. The medical device as set forth in claim 1, characterized in that the acellular biological matrix is of human and/or animal origin.

7. The medical device as set forth in the claim 6, characterized in that the acellular biological matrix of animal origin is selected from among the acellular biological matrices of porcine, bovine, equine, caprine, or fish origin, and mixtures thereof.

8. The medical device as set forth in claim 1, characterized in that the acellular biological matrix is selected from one of the following biological matrices: dermis, intestinal submucosa, aorta, bladder, amniotic membrane, peritoneum, pericardium, dura mater, tendons, bones, cartilage, and mixtures thereof.

9. The medical device as set forth in claim 1, characterized in that the polymer(s) are selected from among the following polymers: polyhydroxyalkanoates, poly(glycolides), poly(lactide-co-glycolides), polylactic acid, polyglycolic acid, poly(lactic acid-coglycolic acids), polycaprolactones, poly(orthoesters), polyanhydrides, poly(phosphazenes), polyesters, poly(lactide-co-caprolactones), polycarbonates; tyrosine polycarbonates, polyamides, polyesteramides, poly(dioxanones), poly(alkylene alkylates), polyethers, polyvinyl pyrrolidones, polyurethanes, polyether esters, polyacetals, polycyanoacrylates, poly(oxyethylene), copolymers of poly(oxypropylene), polyacetals, polyketals, polyphosphates, polyphosphoesters, polyalkylene oxalates, polyalkylene succinates, polymaleic acids, chitin, chitosan, and mixtures thereof.

10. The medical device as set forth in claim 1, characterized in that the polymer(s) are selected from among the following polyhydroxyalkanoates: P4HB, co-polymers, and mixtures thereof.

11. The medical device as set forth in claim 1, characterized in that the acellular biological matrix:
has a residual moisture level of between 10% and 18%, and/or
has a surface appearance improving the adhesion of at least one polymer.

12. The medical device according to claim 11, characterized in that the acellular biological dermal matrix has been obtained by a surface treatment before coating, chosen from a chemical and/or mechanical and/or electrochemical and/or physical surface treatment.

13. A method for manufacturing a medical device as set forth in claim 1, characterized in that it comprises the implementation of the following steps:
preparation of an acellular biological matrix so as to allow the adhesion of a solution comprising at least one polymer, and
partial or total coating of the acellular biological matrix with a solution comprising at least one polymer.

14. The method for manufacturing a medical device as set forth in claim 13, characterized in that the coating is performed at a temperature lower than or equal to the denaturation temperature of the collagen.

15. The method for manufacturing a medical device as set forth in claim 14, characterized in that the coating is performed at a temperature between 10 and 60° C.

16. The method for manufacturing a medical device as set forth in claim 13 ,characterized in that the biological matrix is dried so as to have a residual moisture level of between 10% and 18%.

17. The method for manufacturing a medical device as set forth in claim 13, characterized in that the step of preparing the cellular biological matrix consists of a chemical and/or mechanical and/or electrochemical and/or physical surface treatment.

18. The method for manufacturing a medical device as set forth in claim 13, characterized in that the coating is carried out by pour coating, spray coating and dip coating.

19. The method for manufacturing a medical device as set forth in claim 13, characterized in that the solution comprising at least one polymer was obtained previously through solubilization of the polymer(s) in at least one polar solvent.

20. The method for manufacturing a medical device as set forth in claim 19, characterized in that, after solubilization of the polymer(s), the solution is degassed and/or debubbled under vacuum in order to purge the mixture of air bubbles.

21. The method for manufacturing a medical device as set forth in claim 13, characterized in that the polymer solution comprises at least P4HB dissolved in an acetone solution.

22. A use of a medical device as set forth in claim 1, as is or in altered form as an implant.
